(19) United States
(12) Patent Application Publication
(10) Pub. No.: US 2015/0084630 A1
(43) Pub. Date: Mar. 26, 2015

(54) SYSTEM AND METHOD FOR GENERATING INVASIVELY HYPERPOLARIZED IMAGES

(71) Applicant: ASPECT IMAGING LTD., Shoham (IL)

(72) Inventor: Uri RAPOPORT, Moshav Ben Shemen (IL)

(21) Appl. No.: 14/556,682

(22) Filed: Dec. 1, 2014

Related U.S. Application Data
(62) Division of application No. 13/883,864, filed on May 29, 2013.
(60) Provisional application No. 61/413,991, filed on Nov. 16, 2010.

(51) Int. Cl. G01R 33/54; A61B 5/01; A61B 8/00; A61B 5/00; A61B 5/055; A61B 6/03

(52) U.S. Cl. CPC G01R 33/54; A61B 5/0555; A61B 6/032; A61B 8/40; A61B 5/0071; A61B 5/015; A61B 5/0035
USPC 324/309

(57) ABSTRACT

The present invention includes a system and method for generating images of at least one unhyperpolarized portion of a specimen by indirectly hyperpolarizing the at least one portion by irradiating the unhyperpolarized portion by radiation emitted from the de-excitation of excited nuclei of a hyperpolarized substance. The hyperpolarized substance is located in proximity to the specimen. Typically, the images are generated by an MRI/NMR device.

### FIELD OF THE INVENTION

The present invention relates to a system and method for generating clear anatomical images of regions of interest of a patient or a specimen in-vivo by non-invasively hyperpolarizing the regions of interest of the patient or the specimen.

### BACKGROUND OF THE INVENTION

The following prior art is believed to be the current status of the art: U.S. Pat. No. 5,545,396 to M. S. Albert et al. describes a method for administering a hyperpolarized noble gas to a human or animal subject. The method includes the detection and generation of a spatial distribution of the noble gas in the subject. This prior art requires an invasive injection of the hyperpolarized noble in the patient.

US Published Patent Application No. 2009/0016964 to N. Kalechovsky et al. describes a system and method for hyperpolarizing a solvent material and transferring the hyperpolarization from the solvent to a target material. However, this prior art requires that the solvent and target material are in contact.

U.S. Pat. No. 6,808,699 to B. Drehuys et al. describe a method for evaluating the effects of drug therapy on a patient by the patient inhaling a dosage of polarized ¹²⁹Xe. This method requires the inhalation of ¹²⁹Xe gas.

U.S. Pat. No. 6,008,644 to lb Leunbach et al. describes a method for enhancing an MRI image of a specimen by injecting an compositing including an OMRI (Overhauser MRI) contrast agent and an MRI imaging agent into the specimen.

The cited art methods for enhancing an MRI signal include invasive methods for introducing hyperpolarized substances into a patient or a specimen in order to enhance the MRI image of a region of interest, such as body fats, in the patient or the specimen.

Thus, there is an unmet requirement for generating clear anatomical images of portions of a patient or a specimen in-vivo, by means of a non-invasively hyperpolarizing the region of interest of the patient or specimen.

### SUMMARY OF THE INVENTION

The present invention seeks to present to a system and method for generating clear anatomical images of at least one portion of a plurality of portions of at least one region of interest of a patient or specimen, in-vivo, generated by non-invasively hyperpolarizing the at least one portion of the plurality of portions of at least one region of interest of the patient or the specimen. Typically, the at least one portion of interest includes a region of body fats, such as lipids.

It is known in the art that the certain organs and/or regions of interest in the patient or specimen generate unclear MRI images of regions of interest in the mammalian body. For example, the water protons in lipids are very difficult to detect due to the short T₂ relaxation times in the lipids. Additionally, signals from other biologically interesting nuclides have a low concentration and thus are very difficult to detect.

Known methods for enhancing the Signal-to-Noise Ratio (SNR) of the regions of interest, include introducing hyperpolarized nuclei, such as isotopes ¹³C, ¹⁵N, ¹H, ²H, ³He, ³¹P, ¹⁹F, ²⁹Si and ¹²⁹Xe contained in fluids, into the body of interest. The methods of introducing the hyperpolarized nuclei into the patient or specimen include inhalation of the hyperpolarized gases by the patient or specimen or injecting hyperpolarized fluids into the body of the patient or specimen.

The present invention describes and includes a non-invasive hyperpolarizing system and method for enhancing MRI images of at least one unhyperpolarized portion of at least a plurality of unhyperpolarized portions of at least one region of interest, such as lipids, in the patient or specimen. It is known in the art that various unhyperpolarized regions of the patient or specimen generate anatomically unclear images thereof and thus, impeding the medical diagnosis of the patient or the specimen.

The system and methods of the present invention include accommodating the patient or the specimen within an inner chamber, which is enclosed and encompassed by an outer chamber. The outer chamber contains a hyperpolarized substance, such as water. The hyperpolarized substance hyperpolarizes at least one unhyperpolarized portion of at least one region of interest of the patient or the specimen, such as body fats. It is appreciated that the inner chamber is fluidly impermeable to the outer chamber and thus, the patient or the specimen is fluidly-isolated from the hyperpolarized substance. Thus, there is no fluid contact between the specimen and the hyperpolarized substance.

Typically, the patient or the specimen is selected from the group consisting of a mammalian specimen, a human patient, a premature neonate, a reptile specimen, an amphibian specimen, a rodent specimen, a biological specimen, a biological organ, an amphibian, in vivo biological tissue, in vivo biological tissue organ, ex vivo biological tissue, ex vivo biological organ and any combination thereof.

An RF signal generator generates RF signals, which preferably excite the hyperpolarized nuclei of the hyperpolarized substance. The excited hyperpolarized water nuclei in the hyperpolarized substance de-excite and irradiate the at least one unhyperpolarized portion, thereby hyperpolarizing the at least one of the plurality of unhyperpolarized portions of the patient or the specimen. Preferably, the irradiated energy is in the RF spectrum. The at least one hyperpolarized portion generates radiation, preferably RF radiation, which is detected and analyzed by an imaging device.

The imaging device generates a plurality of images of the at least one hyperpolarized portion of the at least one region of interest.

A typical imaging device is selected from the group consisting of an MRI imaging device, an NMR imaging device, a CT imaging device, an X-ray imaging device, an ultrasound imaging device, a fluorescence imaging device, a thermal imaging device and any combination thereof.

The de-exciting nuclei of the hyperpolarized substance, such as water, irradiates the at least one unhyperpolarized portion of the at least one region of interest, such as the body fats, preferably, by RF radiation. The hyperpolarized substance operates as an RF transmitter and RF antenna. Additionally, due to the larger quantity of the hyperpolarized substance contained in the outer chamber encompassing the inner chamber, the hyperpolarized substance also operates as an RE amplifier.

The hyperpolarization of the unhyperpolarized portion, such as the body fats and lipids, results in an enhancement in a population of polarized nuclei in hyperpolarized portion of the region of interest relative to the population of the polarized nuclei in an unhyperpolarized portion of the region of interest. The at least one hyperpolarized portion of the region of interest generates a signal, preferably in the RF spectrum, which is detected and analyzed by the imaging device.

It is known in the art, that the Signal-to-Noise Ratio (SNR) is proportional to the polarization factor, P:

SNR α P

and P is given by:

$${}_{}^{•}{P}{}_{•}^{} = \frac{{N}^{+} - {N}^{-}}{{N}^{+} + {N}^{-}}$$

where N⁺ is the number of nuclei spins parallel to the magnetic field and

N⁻ is the number of nuclei spins anti-parallel to the magnetic field. Thus, by hyperpolarizing the nuclei in the at least one unhyperpolarized portion of the region of interest, an increase in the SNR is obtained.

Preferably, the specimen is placed within a housing including a fluidly impermeable outer chamber and fluidly impermeable inner chamber. The outer chamber preferably includes hyperpolarized water. The specimen, accommodated within the inner chamber, is fluidly-isolated from the hyperpolarizing substance.

It is appreciated that the inner chamber is coupled to the required life-line connections and systems for maintaining the patient or specimen within the inner chamber in-vivo conditions.

It is further appreciated that the generation of the plurality of images of the at least one hyperpolarized portion of the at least one region of interest is within a time scale, that at least includes the hyperpolarized substance maintains its hyperpolarization status, de-excitation of the excited nuclei of the hyperpolarized substance that hyperpolarizes the unhyperpolarized portion of the region of interest and the detection of the hyperpolarized at least one hyperpolarized portion of the specimen.

There is provided in accordance with a preferred embodiment of the present invention, an indirect-hyperpolarization system including a fluidly-sealable inner chamber accommodating a specimen the specimen includes at least one unhyperpolarized portion of a plurality of unhyperpolarized portions of at least one region of interest and a fluidly-sealable outer chamber encompassing the fluidly-sealable inner chamber and including a hyperpolarized substance. The specimen is fluidly-isolated from the hyperpolarized substance and the hyperpolarized substance hyperpolarizes the at least one unhyperpolarized portion by electromagnetically coupling the hyperpolarized substance and the at least one unhyperpolarized portion.

Further in accordance with a preferred embodiment of the present invention further including an imaging device for generating a plurality of images of the at least one hyperpolarized portion of the specimen the imaging device is selected from the group consisting of an MRI imaging device, an NMR device, a CT imaging device, an X-ray imaging device, an ultrasound imaging device, a fluorescence imaging device, a thermal imaging device and any combination thereof.

Still further in accordance with a preferred embodiment of the present invention the imaging device further detects the presence of the at least one hyperpolarized portion and generates at least one anatomically clear image of the at least one hyperpolarized portion.

Additionally, in accordance with a preferred embodiment of the present invention the hyperpolarized substance includes water.

Moreover in accordance with a preferred embodiment of the present invention the at least one unhyperpolarized portion includes body fats and the at least one hyperpolarized portion includes body fats.

Further in accordance with a preferred embodiment of the present invention the specimen is selected from the group consisting of a mammal specimen, a human specimen, a premature neonate, a reptile specimen, an amphibian specimen, a rodent specimen, a biological specimen, a biological organ, an amphibian, in vivo biological tissue, in vivo biological tissue organ, ex vivo biological tissue, ex vivo biological organ and any combination thereof.

Still further in accordance with a preferred embodiment of the present invention the electromagnetic coupling is generated by electromagnetic signals generated by an electromagnetic signal generator. Preferably, the electromagnetic signal generator includes an RF signal generator generating RF signals.

There is provided in accordance with another preferred embodiment of the present invention an indirect-hyperpolarization system including a fluidly-sealable inner chamber accommodating a specimen the specimen includes at least one unhyperpolarized portion of a plurality of unhyperpolarized portions of at least one region of interest, a fluidly-sealable outer chamber encompassing the fluidly-sealable inner chamber and including a hyperpolarized substance, and an RF signal generator for exciting nuclei of the hyperpolarized substance. The specimen is fluidly-isolated from the hyperpolarized substance and the at least one unhyperpolarized portion is hyperpolarized by electromagnetic signals emitted by de-excited the nuclei such that an imaging device is enabled to detect the presence of the at least one hyperpolarized portion.

Further in accordance with a preferred embodiment of the present invention the electromagnetic signals have a wavelength within the RF spectrum.

Still further in accordance with a preferred embodiment of the present invention the imaging device is selected from the group consisting of an MRI device, an NMR device, a CT device, an X-ray device, an ultrasound device, a fluorescence device, a thermographic device and any combination thereof.

Additionally in accordance with a preferred embodiment of the present invention the imaging device further generates at least one anatomically clear image of the at least one hyperpolarized portion.

Still further in accordance with a preferred embodiment of the present invention the hyperpolarized substance includes water.

Additionally in accordance with a preferred embodiment of the present invention the at least one unhyperpolarized portion includes body fats and the at least one hyperpolarized portion includes body fats.

Furthermore in accordance with a preferred embodiment of the present invention the specimen is selected from the group consisting of a mammal, a human, a premature neonate, a reptile, a sea animal, a rodent, a biological specimen, a biological organ, an amphibian, in vivo biological tissue, in vivo biological tissue organ, ex vivo biological tissue, ex vivo biological organ and any combination thereof.

There is provided in accordance with yet another preferred embodiment of the present invention a system for imaging at least one unhyperpolarized portion of a specimen including a fluidly-sealable inner chamber accommodating the specimen, a fluidly-sealable outer chamber encompassing the fluidly-sealable inner chamber and including a hyperpolarized substance and an imaging device. The specimen is fluidly-isolated from the hyperpolarized substance and the hyperpolarized substance hyperpolarizes the at least one unhyperpolarized portion by electromagnetic coupling the hyperpolarized substance and the at least one unhyperpolarized portion such that the imaging device is enabled to detect the presence of the at least one hyperpolarized portion.

Further in accordance with a preferred embodiment of the present invention the electromagnetic coupling is generated by an RF signal generating system located in proximity to the imaging device and RF signal generating system includes an RF signal generator and an RF antenna.

Still further in accordance with a preferred embodiment of the present invention the RF signal generating system is adapted to excite nuclei of the hyperpolarized substance such that RF radiation emitted by de-excitation of the excited nuclei hyperpolarizes the at least one unhyperpolarized portion.

Additionally in accordance with a preferred embodiment of the present invention the electromagnetic radiation includes RF radiation.

Moreover in accordance with a preferred embodiment of the present invention the system further including at least one RF receiving coil located within the inner chamber and located in proximity to the at least one hyperpolarized portion and adapted to receive RF radiation emitted by the at least one hyperpolarized portion.

Additionally in accordance with a preferred embodiment of the present invention the system further including a displacement system adapted to displace the at least one RF receiving coil to the proximity location and the displacement of the at least one RF receiving coil is selected from the group consisting of a translational displacement parallel to a longitudinal axis of the inner chamber and with an accuracy of at least 3 mm to the at least one hyperpolarized portion and a rotational displacement about the longitudinal axis of the inner chamber and any combination thereof.

Further in accordance with a preferred embodiment of the present invention the imaging system detects and analyzes the radiation emitted by the at least one hyperpolarized portion and the imaging system is adapted to generate a plurality of images of the at least one hyperpolarized portion of the specimen.

Still further in accordance with a preferred embodiment of the present invention the imaging device is selected from the group consisting of an MRI device, an NMR device, an MRI device, a CT device, an X-ray device, an ultrasound device, a fluorescence device, a thermographic device and any combination thereof.

Additionally in accordance with a preferred embodiment of the present invention the specimen is selected from the group consisting of a mammal, a human, a premature neonate, a reptile, a sea animal, a rodent, a biological specimen, a biological organ, an amphibian, in vivo biological tissue, in vivo biological tissue organ, ex vivo biological tissue, ex vivo biological organ and any combination thereof.

There is provided in accordance with yet another preferred embodiment of the present invention a method for imaging at least one unhyperpolarized portion of a specimen including providing an imaging device for generating a plurality of images of at least one hyperpolarized portion of the specimen, locating an indirect-hyperpolarization device within the imaging device, hyperpolarizing the at least one unhyperpolarized portion and generating a plurality of images of the at least one hyperpolarized portion. The indirect-hyperpolarization device includes a fluidly-sealable outer chamber, a fluidly-sealable inner chamber encompassed by the fluidly-sealable outer chamber and accommodating the specimen and the specimen is fluidly-isolated from the hyperpolarized substance.

Further in accordance with yet another preferred embodiment of the present invention further includes locating and aligning the inner chamber within the outer chamber.

Still further in accordance with yet another preferred embodiment of the present invention further including locating an RF signal generating system for generating and transmitting RF energy thereby exciting the nuclei of the hyperpolarized substance and wherein de-excitation of the nuclei hyperpolarizes the at least one unhyperpolarized portion.

### BRIEF DESCRIPTION OF THE DRAWINGS

A preferred embodiment of the current invention is described hereinbelow with reference to the following drawings:

FIG. 1 shows an indirect-hyperpolarization system, in accordance with a preferred embodiment of the present invention;

FIG. 2 shows an imaging system, such as an NMR/MRI imaging system including, inter alia, an indirect-hyperpolarization system, in accordance with another preferred embodiment of the present invention and

FIGS. 3A-3B show a typical flow chart for generating a plurality of images of a region of interest of a specimen, in accordance with another preferred embodiment of the present invention.

### DETAILED DESCRIPTION OF PREFERRED EMBODIMENTS OF THE PRESENT INVENTION

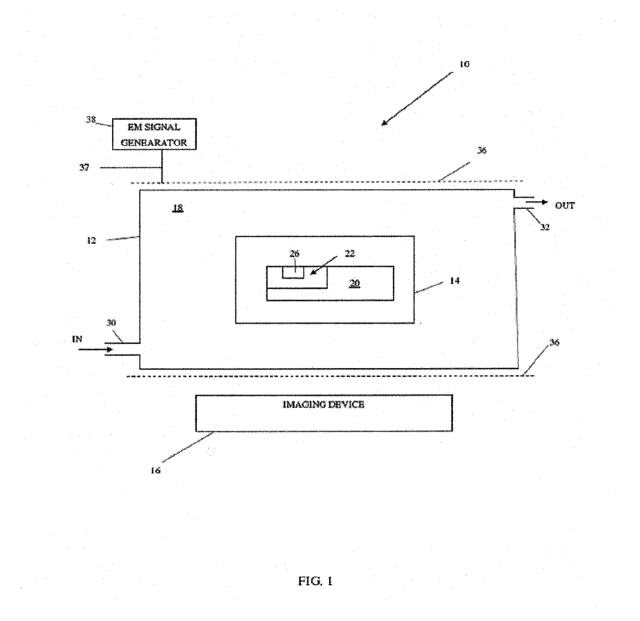

Reference is now made to FIG. 1, which shows an indirect-hyperpolarization system 10, in accordance with a preferred embodiment of the present invention. The indirect-hyperpolarization system 10 includes, inter alia, a fluidly-sealable outer chamber 12 encompassing a fluidly-sealable inner chamber 14.

The outer chamber 12 includes, inter alia, a hyperpolarized medium 18, such as water. The inner chamber 14 is fluidly impermeable from the outer chamber 12, such that the hyperpolarized water 18 is unable to penetrate into the inner chamber 12. The inner chamber 14 is fully immersed within the hyperpolarized water 18, as shown in FIG. 1.

The inner chamber 14 accommodates a specimen 20. The specimen 20 is fluidly-isolated from the hyperpolarized water 18 and the specimen 20 includes at least one region of interest 22. The at least one region of interest 22 includes, inter alia, at least one unhyperpolarized portion 26, such as body fats, of a plurality of unhyperpolarized portions of the at least one region of interest 22. Typically, the body fats 26 include lipids. In the present invention, an imaging device 16 generates at, least one plurality of images 24 of the at least one unhyperpolarized portion 26, thereby assisting in the determination of a medical diagnosis of the specimen 20.

Typically, the specimen 20 is selected from the group consisting of a mammal specimen, a human patient, a premature neonate, a reptile specimen, an amphibian specimen, a rodent specimen, a biological specimen, a biological organ, an amphibian, in vivo biological tissue, in vivo biological tissue organ, ex vivo biological tissue, ex vivo biological organ and any combination thereof.

The imaging system 16 is located in proximity to the indirect-polarization system 10 so as to detect and analyze the plurality of images 24 generated by the hyperpolarized portion 26.

Typically, the imaging device 16 is selected from the group consisting of an MRI imaging device, an NMR imaging device, a CT imaging device, an X-ray imaging device, an ultrasound imaging device, a fluorescence imaging device, a thermal imaging device and any combination thereof. The imaging device 16 generates the plurality of images 24 of the at least one hyperpolarized portion 26.

Water is externally hyperpolarized in order to generate the hyperpolarized water 18, prior to its introduction into the outer chamber 12, as is known in the art.

The hyperpolarized water 18 is introduced into the outer chamber 12 via an inlet port 30 and fluidly sealed within the outer chamber 12. The outer chamber 12 also includes an outlet port 34 to allow draining of the hyperpolarized water 18 from the outer chamber 12. It is appreciated that replenishing the hyperpolarized water 18 in the outer chamber 12 maintains the required quantity of hyperpolarized water 18 for the imaging process of the at least one unhyperpolarized portion 26.

An RF transmitter 36, typically in the form of a coil, typically encompasses the outer chamber 12 and is electronically coupled by an electronic coupling device 37, such as a waveguide, to an electromagnetic (EM) signal generator 38. The EM signal generator 38 generates RF signals which are transmitted by means of the RF transmitting coil 36 to the hyperpolarized water 18 thereby exciting the hyperpolarized water nuclei therein.

Additionally or alternatively, an RF antenna is coupled to the EM signal generator 38 by means of a suitable coupling device, for radiating the RF signals to the hyperpolarized water 18.

The excited hyperpolarized water 18 de-excites and transmits radiation, preferably RF radiation, which irradiates the region of interest 22. The at least one unhyperpolarized portion 26, contained within the at least one region of interest 22, is hyperpolarized and the at least one hyperpolarized portion 26 emits radiation, preferably, in the RF spectrum. The emitted radiation is detected by the imaging device 16 and the imaging device generates the plurality of images 24.

It is appreciated that the hyperpolarized water 18 operates as an RF transmitter and an RF antenna and the radiation emitted by the de-excitation of the excited water irradiates the at least one unhyperpolarized portion 26. The unhyperpolarized portion 26 is hyperpolarized by the electromagnetic coupling between the hyperpolarized water 18 and the at least one unhyperpolarized portion 26, as is known in the art.

In addition, due to the larger quantity of the hyperpolarized water 18, the hyperpolarized water 18 also operates as an RF amplifier.

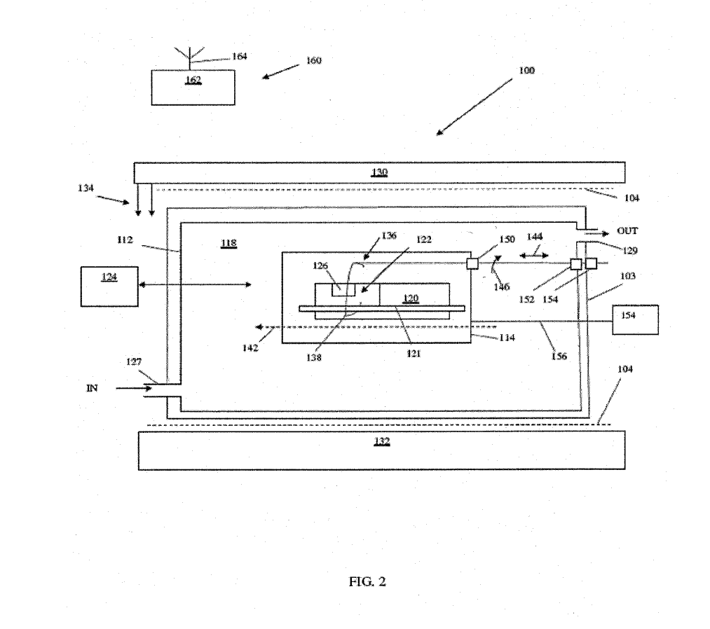

Reference is now made to FIG. 2, which shows an imaging system 100, such as an NMR/MRI imaging system including, inter alia, an indirect-hyperpolarization system 102, in accordance with another preferred embodiment of the present invention. The imaging system 100 includes, inter alia, an RF transmitting coil 104 for MRI imaging, which is located within the imaging system 100 and encompasses the indirect-hyperpolarization system 102.

It is appreciated that the indirect-hyperpolarization system 102 is of similar construction and operation as the indirect-hyperpolarization system 10 described hereinabove.

The indirect-hyperpolarization system 102 includes, inter alia, a fluidly-sealable housing 103. The fluidly-sealable housing 103 includes, inter alia, a fluidly-sealable outer chamber 112 enclosing and encompassing a fluidly-sealable inner chamber 114.

The outer chamber 112 includes, inter alia, a hyperpolarized substance 118, such as water. The inner chamber 114 is fluidly impermeable from the outer chamber 112, such that the substance 118 is unable to penetrate into the inner chamber 114.

The inner chamber 114 accommodates a specimen 120, which is located on a translationally and/or rotationally displaceable platform 121 for inserting and locating the specimen 120 within the inner chamber 114, as is known in the art.

The specimen 120 includes at least one region of interest 122. The at least one region of interest 122 includes at least one unhyperpolarized portion 126 of a plurality of unhyperpolarized portions, such as body fats, for which it is required to generate at least one plurality of images 124 thereby assisting in the determination of a medical diagnosis of the specimen 120.

It is appreciated that the specimen 120 is fluidly-isolated from the hyperpolarizing substance 118. The region of interest 122 includes, inter alia, at least one region of unhyperpolarized body fats 126, such as lipids.

Typically, the specimen 120 is selected from the group consisting of a mammal specimen, a human specimen, a premature neonate, a reptile specimen, an amphibian specimen, a rodent specimen, a biological specimen, a biological organ, an amphibian, in vivo biological tissue, in vivo biological tissue organ, ex vivo biological tissue, ex vivo biological organ and any combination thereof.

The water in the outer chamber 112 is hyperpolarized prior to its introduction into the outer chamber 112, as is known in the art.

The hyperpolarized water 118 is introduced into the outer chamber 112 via an inlet port 127 and exits the outer chamber 112 via an output port 129. Thus, it is possible to replenish the amount of the hyperpolarized water 118 thereby ensuring that the hyperpolarized water 118 remains hyperpolarized throughout the imaging process.

It is appreciated that the hyperpolarized water 118 encloses and encompasses the inner chamber 114 and the hyperpolarized water 118 is stored within the outer chamber 112. It is further appreciated that the specimen 122 is fluidly-isolated from the hyperpolarized water 118.

An electromagnetic (EM) signal generating system 160 is located in proximity to the housing 103. The EM generating system 160 includes, inter alia, an RF signal generator 162 and an RF antenna 164. The RF signal generator 162 generates RF signals which irradiate the hyperpolarized water 118 thereby exciting the hyperpolarized water nuclei therein.

The nuclei in the hyperpolarized water 118 de-excite and irradiate the at least one unhyperpolarized portion 126 hyperpolarizing the at least one unhyperpolarized portion 126.

The RF energy is absorbed by the nuclei of the at least one unhyperpolarized portion 126 by the electromagnetic coupling between the hyperpolarized water 118 and the at least one unhyperpolarized portion 126, as is known in the art.

It is appreciated that the hyperpolarized water 118 operates as an RF transmitter and an RF antenna and the radiation emitted by the de-excitation of the excited water nuclei irradiates the region of interest 122. The at least one unhyperpolarized portion 126 and the unhyperpolarized portion 126 are hyperpolarized by electromagnetic coupling with the hyperpolarized water 118.

It is appreciated that due to the larger quantity of the hyperpolarized water 118, relative to the quantity of unhyperpolarized portion 126, the hyperpolarized water 118 also operates as an RF signal amplifier.

The imaging system 100, such as an NMR/MRI imaging system, encloses and encompasses the housing 103 as well as the indirect-hyperpolarization system 102 so as to detect and analyze the plurality of images 124 generated by hyperpolarizing of the at least one plurality of unhyperpolarized portions 126.

The MRI/NMR imaging system 100 further includes, inter alia, at least two magnets 130 and 132 for generating a uniform magnetic field 134 therebetween. It is appreciated that the system 100 also includes shim coils and other devices for generating the uniform magnetic field 134, as is known in the art.

The radiation emitted by the hyperpolarized portion 126 is detected and analyzed by the MRI/NMR imaging system 100. The imaging device 100 generates the at least one plurality of images 124 of the at least one hyperpolarized portion 126.

The imaging system 100 includes, inter alia, an RF receiving coil system 136, which is located within the inner chamber 114. The RF receiving coil system 136 includes, inter alia, at least one RF receiving coil 138 and a displacement system 140 for displacing the at least one RF receiving coil 138 to a fixed location within the inner chamber 114 for receiving RF radiation emitted by the hyperpolarized body fats 126. The at least one receiver coil 138 is displaced translationally and/or rotationally about a longitudinal axis 142 of the inner chamber 114. The translation and rotational displacements are indicated by arrows 144 and 146, respectively. The translation and/or rotation displacements of the at least one receiver coil 138 enables the imaging system 100 to generate at least one plurality of images 124 of the hyperpolarized body fats 126 at the required translational and rotational locations. The translational displacement typically has an accuracy of at least 3 mm, thus, enabling the RF receiving coil 138 to be accurately located in proximity to the hyperpolarized region 126.

The displacing system 140 and the RF receiver coil 138 are fluidly isolated from the hyperpolarized water 118 by fluid isolating sealing units 150 and 152, as is known in the art. A fluid sealing unit 154 isolates the RF receiver coil 138 and the displacing system 140 from the external environment, as is known in the art. The fluid sealing units 150, 152 and 154 allow an external operator to translationally and/or rotationally displace the RF receiver coil system 136 without disturbing the fluid impermeability of the inner chamber 114 and the fluidly-isolated of the specimen 120 from the hyperpolarized water 118.

The congenial environment is maintained within the inner chamber 114 by an air conditioning system 154 and coupled to the inner chamber 114 via an environmental coupling system 156, as is known in the art.

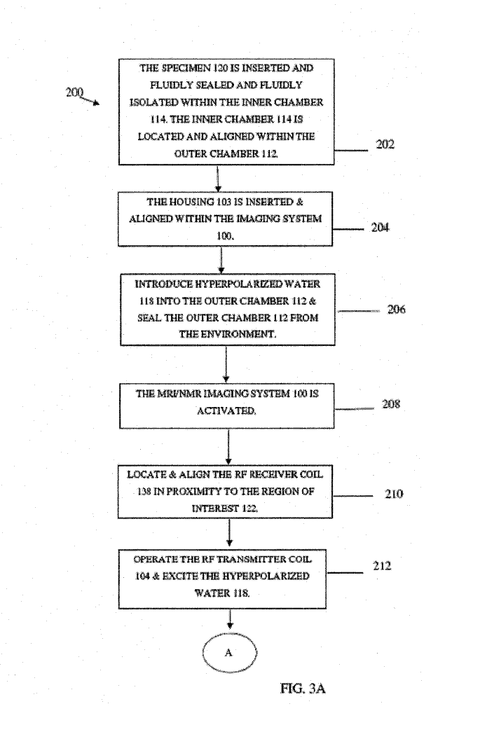

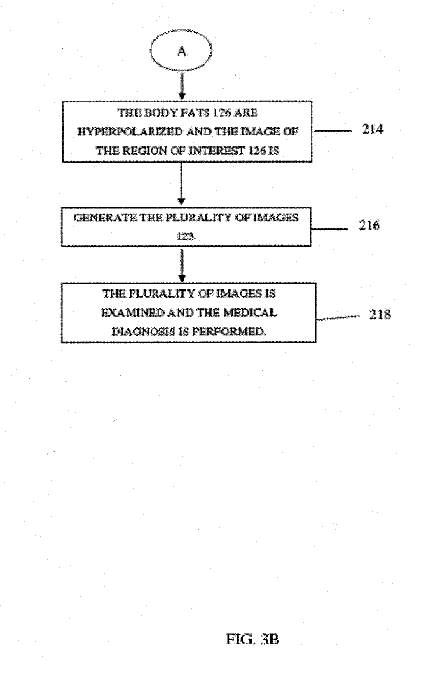

Reference is now made to FIGS. 3A-3B, which show a typical flow chart 200 for generating the at least one plurality of images 124 of the at least one plurality of unhyperpolarized portions 126 of the at least region of interest 122, in accordance with another preferred embodiment of the present invention.

In step 202, the specimen 120 is inserted and fluidly-sealed within the inner chamber 114. The inner chamber 114 is carefully located and aligned within the outer chamber 112, such that the translational and rotational locations of the receiving coil displacement system 140 are aligned relative to the outer chamber 112. The outer chamber 112 is inserted within the housing 103.

In step 204, the housing 103 is inserted and aligned within the imaging system 100.

In step 206, the hyperpolarized water 118 is introduced into the outer chamber 112 and the outer chamber 112 is fluidly sealed from the environment.

In step 208, the MRI/NMR imaging system 100 is activated and the operation of the generation of the plurality of images 124 commences with the generation of the magnetic field 134.

In step 210, the RF receiver coil 138 is located and aligned in proximity to the unhyperpolarized portion 126 of the region of interest 122.

In step 212, the RF transmitter coil 104 is operated and the excitation of the hyperpolarized water 118 commences.

In step 214, the at least one of unhyperpolarized portions 126 the plurality of unhyperpolarized portions is hyperpolarized.

In step 216, the images of the at least one plurality of unhyperpolarized portion 126 is generated.

In step 218, the plurality of images is examined and the medical diagnosis is performed.

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

What is claimed is:

1. A method for imaging at least one unhyperpolarized portion of a specimen comprising:
providing an imaging device for generating a plurality of images of at least one hyperpolarized portion of said specimen;
locating an indirect-hyperpolarization device within said imaging device, said indirect-hyperpolarization device comprises:
a fluidly-sealable outer chamber;
a fluidly-sealable inner chamber encompassed by said fluidly-sealable outer chamber and accommodating said specimen wherein said specimen is fluidly-isolated from said hyperpolarized substance, and
hyperpolarizing said at least one unhyperpolarized portion;
generating a plurality of images of said at least one hyperpolarized portion.

2. The method for imaging at least one unhyperpolarized portion of a specimen comprising according to claim 1, further comprising locating and aligning said inner chamber within said outer chamber.

3. The method for imaging at least one unhyperpolarized portion of a specimen comprising according to claim 2, further comprising locating an RF signal generating system for generating and transmitting RF energy thereby exciting the nuclei of said hyperpolarized substance and wherein de-excitation of said nuclei hyperpolarizes said at least one unhyperpolarized portion.

4. The method for imaging at least one unhyperpolarized portion of a specimen comprising according to claim 3, further comprising locating an RF receiving transmitting coil in proximity to said at least one hyperpolarized portion for detecting RF radiation emitted by said at least one hyperpolarized portion.
